(19) United States
(12) Patent Application Publication
(10) Pub. No.: US 2024/0023936 A1
(43) Pub. Date: Jan. 25, 2024

(54) ULTRASOUND ENDOSCOPE

(71) Applicant: OLYMPUS MEDICAL SYSTEMS CORP., Tokyo (JP)

(72) Inventors: Ren TAKEUCHI, Tokyo (JP); Takayuki KONDO, Tokyo (JP)

(73) Assignee: OLYMPUS MEDICAL SYSTEMS CORP., Tokyo (JP)

(21) Appl. No.: 17/869,748

(22) Filed: Jul. 20, 2022

(51) Int. Cl. A61B 8/00; A61B 8/12; B06B 1/06

(52) U.S. Cl. CPC A61B 8/4483; A61B 8/12; B06B 1/067; B06B 2201/76

(57) ABSTRACT

An ultrasound endoscope includes: an ultrasound transducer including a piezoelectric element that includes a plurality of grooves with a groove filling material filled in the plurality of grooves, the groove filling material including the following (1) to (3):
(1) an epoxy resin;
(2) at least one filler selected from the group consisting of diamond, aluminum oxide, silicon oxide, silicon carbide, boron carbide, iron oxide (III), chromium oxide (III), and cubic boron nitride; and
(3) an aliphatic diol diglycidyl ether.

### BACKGROUND

### 1. Technical Field

The present disclosure relates to an ultrasound endoscope.

### 2. Related Art

In the related art, there has been known an ultrasound endoscope in which an ultrasound transducer is disposed at a distal end of an insertion portion to be inserted into a body of a subject.

The ultrasound transducer is schematically constituted by a piezoelectric element, an acoustic matching layer and an acoustic lens disposed on one surface of the piezoelectric element, and a backing material disposed on another surface of the piezoelectric element. The piezoelectric element has a plurality of grooves each having a width of several tens μm, and the grooves are filled with a groove filling material for improving acoustic characteristics.

On the other hand, there has been proposed an ultrasound probe having a connector that connects gaps between a plurality of ultrasound transducers arranged in parallel, in which the connector contains a polymer material and a filler (for example, JP 5230248 B2).

In an ultrasound endoscope, a groove filling material that improves acoustic characteristics and has an excellent filling property is desired.

### SUMMARY

In some embodiments, an ultrasound endoscope includes: an ultrasound transducer including a piezoelectric element that includes a plurality of grooves with a groove filling material filled in the plurality of grooves. The groove filling material includes the following (1) to (3):
(1) an epoxy resin;
(2) at least one filler selected from the group consisting of diamond, aluminum oxide, silicon oxide, silicon carbide, boron carbide, iron oxide (III), chromium oxide (III), and cubic boron nitride; and
(3) an aliphatic diol diglycidyl ether.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of an endoscope system including an ultrasound endoscope according to an embodiment; and

FIG. 2 is a side view illustrating an ultrasound transducer used in the ultrasound endoscope of FIG. 1.

### DETAILED DESCRIPTION

Hereinafter, an embodiment will be described with reference to the attached drawings.

### Embodiment

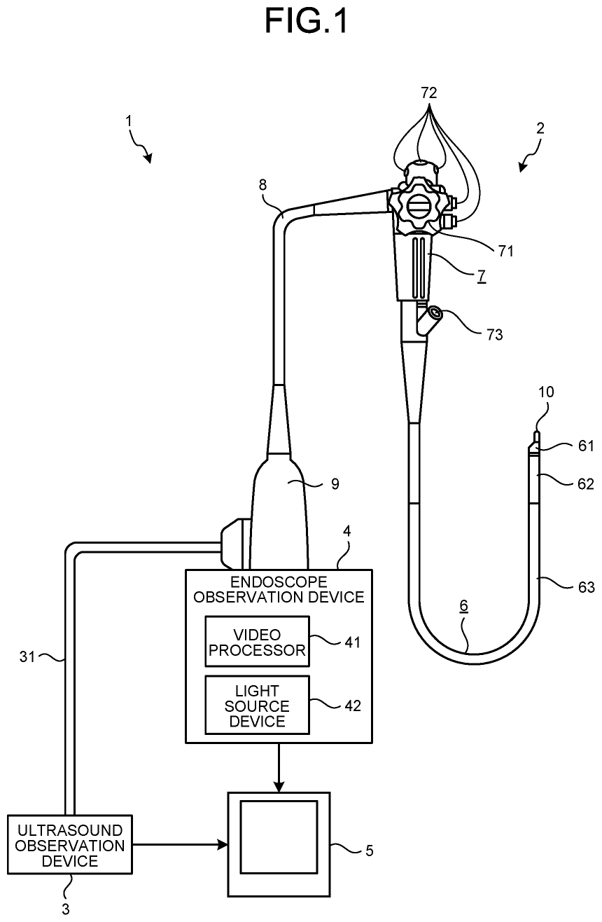

FIG. 1 is a schematic diagram illustrating a schematic configuration of an endoscope system including an ultrasound endoscope according to an embodiment. An endoscope system 1 is a system that performs ultrasound diagnosis and treatment in a subject such as a human using an ultrasound endoscope 2. As illustrated in FIG. 1, the endoscope system 1 includes the ultrasound endoscope 2, an ultrasound observation device 3, an endoscope observation device 4, and a display device 5.

The ultrasound endoscope 2 can be partially inserted into a subject, and has a function of transmitting an ultrasound pulse (acoustic pulse) toward a body wall in the subject, receiving an ultrasound echo reflected by the subject, and outputting an echo signal, and a function of imaging the inside of the subject and outputting an image signal. Note that a detailed configuration of the ultrasound endoscope 2 will be described later.

The ultrasound observation device 3 is electrically connected to the ultrasound endoscope 2 via a cable 31, outputs a pulse signal to the ultrasound endoscope 2 via the cable 31, and receives an echo signal as an input from the ultrasound endoscope 2. Then, the ultrasound observation device 3 performs predetermined processing on the echo signal to generate an ultrasound image.

To the endoscope observation device 4, an endoscope connector 9, which will be described later, of the ultrasound endoscope 2 is detachably connected. The endoscope observation device 4 includes a video processor 41 and a light source device 42.

The video processor 41 receives an image signal from the ultrasound endoscope 2 as an input via the endoscope connector 9. Then, the video processor 41 performs predetermined processing on the image signal to generate an endoscopic image.

The light source device 42 supplies illumination light for illuminating the inside of a subject to the ultrasound endoscope 2 via the endoscope connector 9.

The display device 5 is constituted by a liquid crystal, an organic electro luminescence (EL), a cathode ray tube (CRT), or a projector, and displays an ultrasound image generated by the ultrasound observation device 3, an endoscopic image generated by the endoscope observation device 4, or the like.

### Configuration of Ultrasound Endoscope

Next, a configuration of the ultrasound endoscope 2 will be described. The ultrasound endoscope 2 includes an insertion portion 6, an operating portion 7, a universal cord 8, and the endoscope connector 9.

The insertion portion 6 is a portion to be inserted into a subject. The insertion portion 6 includes an ultrasound probe 10 disposed on a distal end side of the insertion portion 6, a rigid member 61 connected to a proximal end side of the ultrasound probe 10, a bendable portion 62 connected to a proximal end side of the rigid member 61 and capable of being bent, and a flexible tube 63 connected to a proximal end side of the bendable portion 62 and having flexibility.

The operating portion 7 is a portion that is connected to a proximal end side of the insertion portion 6 and receives various operations from a doctor or the like. The operating portion 7 includes a bending knob 71 for bending and operating the bendable portion 62 and a plurality of operating members 72 for performing various operations. The operating portion 7 also has a treatment tool insertion port 73 through which a treatment tool is inserted.

The universal cord 8 is a cord extending from the operating portion 7 and including a light guide, a transducer cable, a signal cable, and a tube constituting a part of a pipeline.

The endoscope connector 9 is disposed at an end of the universal cord 8. Then, the endoscope connector 9 is connected to the ultrasound cable 31 and is inserted into the endoscope observation device 4 to be connected to the video processor 41 and the light source device 42.

### Configuration of Ultrasound Probe

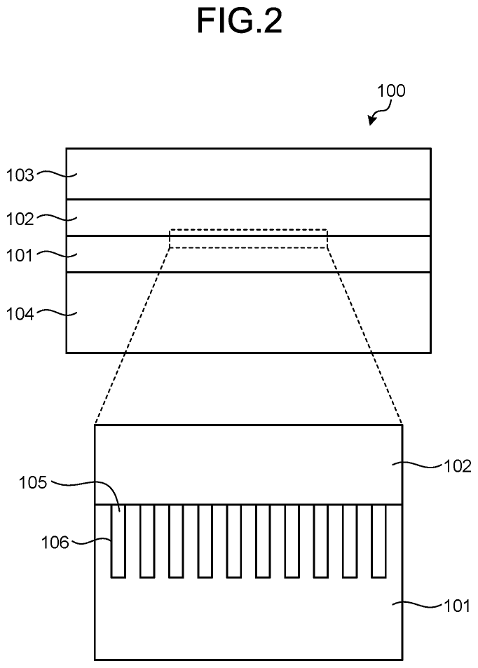

The ultrasound probe 10 includes a plurality of strip-shaped ultrasound transducers 100. FIG. 2 is a side view illustrating the ultrasound transducer. The ultrasound transducer 100 includes a piezoelectric element 101, an acoustic matching layer 102, an acoustic lens 103, and a backing layer 104. Note that the ultrasound transducer 100 only needs to include the piezoelectric element 101, and may have any configuration of a convex type, a linear type, and a radial type.

The piezoelectric element 101 transmits and receives ultrasound to and from a subject. The piezoelectric element 101 may include a piezoelectric body made of a single crystal. Specifically, the piezoelectric element 101 is formed of a piezoelectric material such as a PMN-PT single crystal, a PMN-PZT single crystal, a PZN-PT single crystal, a PIN-PZN-PT single crystal, or a relaxer-based material. Note that the PMN-PT single crystal is an abbreviation of a solid solution of lead magnesium niobate and lead titanate. The PMN-PZT single crystal is an abbreviation of a solid solution of lead magnesium niobate and lead zirconate titanate. The PZN-PT single crystal is an abbreviation of a solid solution of lead zinc niobate and lead titanate. The PIN-PZN-PT single crystal is an abbreviation of a solid solution of lead indium niobate, lead zinc niobate, and lead titanate. The relaxer-based material is a generic term for a three component-based piezoelectric material obtained by adding lead-based composite perovskite, which is a relaxer-based material, to lead zirconate titanate (PZT) for the purpose of increasing a piezoelectric constant and a dielectric constant. The lead-based composite perovskite is represented by Pb(B1,B2)O₃, in which B1 represents any one of magnesium, zinc, indium, and scandium, and B2 represents any one of niobium, tantalum, and tungsten. These piezoelectric materials have an excellent piezoelectric effect. Therefore, even if the size is reduced, an electrical impedance value can be reduced, which is preferable from a viewpoint of impedance matching with an electrode.

In addition, the piezoelectric element 101 has a plurality of grooves 106 on a surface of the piezoelectric element 101 in contact with the acoustic matching layer 102, and the grooves 106 are filled with a groove filling material 105. Details of the groove filling material 105 will be described later.

The acoustic matching layer 102 matches acoustic impedance of the piezoelectric element 101 with acoustic impedance of an observation target for efficient transmission of sound (ultrasound) between the piezoelectric element 101 and the observation target. In the present embodiment, the acoustic matching layer 102 is constituted as one layer, but two acoustic matching layers may be included.

The acoustic lens 103 is made of a material that transmits ultrasound, and converges or diverges the ultrasound. The acoustic lens 103 can be arbitrarily disposed, and the acoustic lens 103 does not have to be included.

The backing layer 104 is formed of a backing material that absorbs and attenuates unnecessary ultrasound generated by operation of the piezoelectric element 101 so as not to return the unnecessary ultrasound to the element.

### Groove Filling Material

The groove filling material contains (1) a resin component, (2) at least one filler selected from the group consisting of diamond, aluminum oxide, silicon oxide, silicon carbide, boron carbide, iron oxide (III), chromium oxide (III), and cubic boron nitride, and (3) an aliphatic diol diglycidyl ether.

The resin component as a main component of the groove filling material is preferably an epoxy resin. As long as the resin component is an epoxy resin, the type of the epoxy resin is not limited, but an epoxy resin having a low viscosity can be preferably used from a viewpoint of easy filling of the resin into the groove.

The filler used for the groove filling material is preferably a highly elastic filler from a viewpoint of improving acoustic characteristics, and is at least one selected from the group consisting of diamond, aluminum oxide, silicon oxide, silicon carbide, boron carbide, iron oxide (III), chromium oxide (III), and cubic boron nitride. Diamond is particularly preferable because diamond has high elasticity, can improve acoustic characteristics even in a small amount, and is excellent in compatibility with an epoxy resin.

In addition, the particle size of the filler to be used can be appropriately determined depending on the width of the groove, but is preferably 20 μm or less.

The groove filling material contains an aliphatic diol diglycidyl ether. By containing the aliphatic diol diglycidyl ether, the fluidity of the groove filling material is improved, and filling into the groove is facilitated. In addition, since the aliphatic diol diglycidyl ether has a functional group capable of reacting with a curing agent of an epoxy resin, the aliphatic diol diglycidyl ether does not impair an elastic modulus even after curing of the epoxy resin, and has a small influence on the acoustic characteristics, which is preferable.

Examples of the aliphatic diol diglycidyl ether include ethylene glycol diglycidyl ether, 1,3-propanediol diglycidyl ether, 1,4-butanediol diglycidyl ether, 1,5-pentanediol diglycidyl ether, and 1,6-hexanediol diglycidyl ether. 1,6-Hexanediol diglycidyl ether is particularly preferable.

The groove filling material preferably contains 10 to parts by mass of the (2) filler and 10 to 50 parts by mass of the (3) aliphatic diol diglycidyl ether with respect to 100 parts by mass of the (1) epoxy resin. When the groove filling material contains the epoxy resin, the filler, and the aliphatic diol diglycidyl ether in the above ratio, acoustic characteristics are improved, and filling into the groove is facilitated.

The groove filling material may contain an epoxy resin curing agent and a silane coupling agent in addition to the epoxy resin, the filler, and the aliphatic diol diglycidyl ether.

When the groove filling material contains the silane coupling agent, wettability with the piezoelectric element can be improved, and filling into the groove is further facilitated.

The groove filling material preferably contains the silane coupling agent in a ratio of 0.1 to 5 parts by mass with respect to 100 parts by mass of the epoxy resin. When the groove filling material contains the silane coupling agent, since the blending amount of the silane coupling agent is small, an influence on the acoustic characteristics is small. However, in order to further reduce the influence on the acoustic characteristics, the silane coupling agent preferably has a functional group that reacts with an epoxy resin curing agent.

### Examples

Groove filling materials prepared in the blending ratios illustrated in the following Table were each poured into a piezoelectric element having a plurality of grooves each having a width of 20 μm from one end side of the groove, and filling properties thereof were confirmed. In addition, after the filling, the groove filling materials were cured, and the acoustic characteristics of the piezoelectric elements were evaluated.

The filling property was evaluated in three grades of “++” (no problem), “+” (filling is performed but it takes time), and “−” (complete filling cannot be performed). In addition, the acoustic characteristics were evaluated in three grades of “++” (target±less than 5%), “+” (target±5% to 15%), and “−” (larger or smaller than target±15%).

TABLE 1

| | | | Com- | Com- | Com- | Com- | Com- | | | | | | | |
|---|---|---|---|---|---|---|---|---|---|---|---|---|---|---|
| | | | parative | parative | parative | parative | parative | Ex- | Ex- | Ex- | Ex- | Ex- | Ex- | Ex- |
| | | | Ex- | Ex- | Ex- | Ex- | Ex- | ample | ample | ample | ample | ample | ample | ample |
| | | | ample 1 | ample 2 | ample 4 | ample 5 | ample 6 | 1 | 2 | 3 | 4 | 5 | 6 | 7 |
| Blend- | Base | Epoxy resin | 100 | 100 | 100 | 100 | — | 100 | 100 | 100 | 100 | 100 | 100 | 100 |
| ing | resin | Silicone resin | — | — | — | — | 100 | — | — | — | — | — | — | — |
| | Additive | Diamond | — | 30 | — | — | 30 | — | 5 | 30 | 30 | 30 | 30 | 30 |
| | | having a | | | | | | | | | | | | |
| | | particle size | | | | | | | | | | | | |
| | | of 20 μm or | | | | | | | | | | | | |
| | | less | | | | | | | | | | | | |
| | | Diamond | — | — | — | — | — | 30 | — | — | — | — | — | — |
| | | having a | | | | | | | | | | | | |
| | | particle size | | | | | | | | | | | | |
| | | of 20 μm or | | | | | | | | | | | | |
| | | more | | | | | | | | | | | | |
| | | 1,6- | — | — | 30 | — | 30 | 30 | 30 | 5 | 60 | 30 | 30 | 30 |
| | | Hexanediol | | | | | | | | | | | | |
| | | diglycidyl | | | | | | | | | | | | |
| | | ether | | | | | | | | | | | | |
| | | Silane | — | — | — | 1 | 1 | 1 | 1 | 1 | 1 | 0.05 | 7 | 1 |
| | | coupling agent | | | | | | | | | | | | |

| | | | | | | | | | | | | | |
|---|---|---|---|---|---|---|---|---|---|---|---|---|---|
| Result | Acoustic | — | ++ | — | — | — | ++ | + | ++ | + | ++ | + | ++ |
| | characteristics | | | | | | | | | | | | |
| | Filling | ++ | — | ++ | ++ | — | + | ++ | + | ++ | + | ++ | ++ |
| | property | | | | | | | | | | | | |

As illustrated in Table 1, it has been confirmed that the groove filling materials of Examples 1 to 7 each containing an epoxy resin, a diamond filler, and 1,6-hexanediol diglycidyl ether have excellent acoustic characteristics and improved filling properties.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

1. An ultrasound endoscope, comprising:
an ultrasound transducer including a piezoelectric element that includes a plurality of grooves with a groove filling material filled in the plurality of grooves,
the groove filling material including the following (1) to (3):
(1) an epoxy resin;
(2) at least one filler selected from the group consisting of diamond, aluminum oxide, silicon oxide, silicon carbide, boron carbide, iron oxide (III), chromium oxide (III), and cubic boron nitride; and
(3) an aliphatic diol diglycidyl ether, wherein the aliphatic diol diglycidyl is 1,6-hexanediol diglycidyl ether.

2. The ultrasound endoscope according to claim 1, wherein the (2) filler is made of diamond.

3. The ultrasound endoscope according to claim 1, wherein the (2) filler has a particle size of 20 μm or less.

4. The ultrasound endoscope according to claim 1, wherein the groove filling material includes 10 to 50 parts by mass of the (2) filler and 10 to 50 parts by mass of the (3) aliphatic diol diglycidyl ether with respect to 100 parts by mass of the (1) epoxy resin.

5. (canceled)

6. The ultrasound endoscope according to claim 1, wherein the groove filling material further includes (4) a silane coupling agent.

7. The ultrasound endoscope according to claim 6, wherein the groove filling material comprises 0.1 to 5 parts by mass of the (4) silane coupling agent with respect to 100 parts by mass of the (1) epoxy resin.
